(19) United States
(12) Patent Application Publication
(10) Pub. No.: US 2022/0034919 A1
(43) Pub. Date: Feb. 3, 2022

(54) System and Method of Automatically Preparing and Analyzing Urine Samples for Identifying Cancer Cells

(71) Applicant: Alfredo R. Zarate, Bethesda, MD (US)

(72) Inventor: Alfredo R. Zarate, Bethesda, MD (US)

(21) Appl. No.: 16/945,278

(22) Filed: Jul. 31, 2020

(51) Int. Cl. G01N 35/00; G01N 1/40; G01N 1/30; G01N 33/493; G01N 15/10; G06T 7/00

(52) U.S. Cl. CPC G01N 35/0099; G01N 1/4077; G01N 35/00732; G01N 1/30; G01N 33/493; G01N 15/10; G06T 2207/30084; G01N 2800/7028; G01N 2001/4083; G01N 2015/1006; G06T 2207/30096; G06T 2207/30024; G06T 2207/10061; G06T 7/0012

(57) ABSTRACT

A system and method of automatically preparing and analyzing urine samples for identifying cancer cells is able to complete conventional diagnostic tasks without lab technicians, cytopathologists, or other medical professionals. The method is provided with at least one source sample, at least one manipulator arm, at least one centrifuge, at least one electronic microscope, and at least one unitary controller. The method is further provided with a cytopathological index containing a visual characteristic database and identification confidence threshold rubrics supporting the automation of visual analyses typically performed manually with a conventional microscope. This method is further provided with a data processing function, wherein data stemming from multiple testing cycles may be collated, formatted, and presented for use by medical professionals in determining and projecting the effectiveness of a course of treatment.

### FIELD OF THE INVENTION

The present invention relates generally to the field of urinalysis and cytopathological assessment methods. More specifically, the present invention recites new means and methods for examination of urine samples to identify cancer cells and other cells using digital image recognition. The proposed system associated with the methods described herein will further support the automatic preparation of raw samples using conventional laboratory processes, integral to an automated data collation and report generation system.

### BACKGROUND OF THE INVENTION

Kidney cancer is among the most common cancers in both men and women today, with occurrence rates rising steadily over the past several decades. It is estimated that in 2019 about 74,000 kidney and renal pelvis cancers were diagnosed and that about 15,000 people will ultimately die from this disease, or related comorbidities. Men have a lifetime risk for developing kidney cancer of 1 in 48 and women of 1 in 83. Likewise, bladder cancer is the ninth most frequently diagnosed cancer worldwide with more than 550,000 cases are diagnosed annually.

Urine cytology is part of the standard triad of diagnostic processes employed to identify renal and urothelial carcinoma, along with cystoscopy and imaging studies. However, these studies are usually only performed whenever there is a clinical evidence of disease, usually hematuria or lower urinary tract symptoms. In addition, the cytological examination of urine samples and measuring soluble or cell attached cancer biomarkers therein offers useful insight into a patient's condition and prognosis. Though these processes are all useful tools for the diagnosis of carcinoma of the kidney and the urinary tract, they are time-consuming and labor-intensive methods. The procedure to prepare the cytology samples may vary between different labs, i.e. volume of sample; duration, rotation rate, and method of centrifugation; and sample analysis standards may differ. Despite any process variance, the samples must eventually be examined by cytopathologists (or by a cytogeneticist in some methods) that may have different degrees of expertise. Most critically, all of them have a high cost and cannot be used to screen a large number of samples.

The Paris system standardizes the urine cytology reporting and increases the sensitivity of diagnosis of High Grade Urothelial Carcinoma (HGUC) by reducing the rate of indeterminate atypical diagnoses. However, it may increase the cases in the atypical category and there is inter-observer variability of findings to contend with in practice. Other methods, e.g. the FISH method (Fluorescent In-Situ Hybridization) have been found to have higher sensitivity than standard cytology using the Papanicolaou (‘Pap’) method for low-grade urothelial carcinoma (UC), or at least comparable sensitivity. Newer tests have been developed, i.e. using CellDetect staining, Hemocolor staining, and measuring other urine biomarkers, i.e. ImmunoCyt (CEA), NMP22 (Nuclear Matrix Protein 22), and UroVysion (chromosomal changes); but they are also cumbersome and expensive. Besides any cost limitations, there is variability in the preparation of the cytology sample (i.e. volume of urine), the methods of processing the sample, (i.e. ThinkPrep, SurePath), and centrifugation (i.e. Cytospin).

Though urine cytology has low sensitivity to diagnose renal cell carcinoma (a higher sensitivity and specificity to diagnose high growth urothelial carcinoma but a lower sensitivity to diagnose low grade tumors), the likelihood of these diagnoses will improve in patients in whom the test is performed along with a urinalysis (UA). The UA is one of the most frequently used medical diagnostic tests; in the US in 1981 it was performed in 50 million occasions among 150 million outpatient visits, in 2016 it was likely done in a higher number (as high as 600 million tests, among 990 million of outpatient visits). In many patients, the UA process is periodically performed over many years for other medical reasons tangential to any known carcinomas. It is proposed that the sensitivity of the test could be improved by centrifuging a larger sample of urine, e.g. 50 L-100 mL, though this would require a reduction in testing costs to make such an approach practical. Further, if the new method utilizing a larger sample is employed alongside a routine conventional UA, the combined findings would be mutually complimentary. Abundant transitional epithelial cells are rarely seen in a urinalysis sediment and presence of same requires a physician to rule out neoplasia or urinary tract infections. Or if red blood cells are identified in the urinalysis sediment, either dysmorphic or isomorphic, a cytological examination to identify malignance cells would be simultaneously performed. This could be accomplished if the cytological examination is performed automatically, at low cost, in large testing batches of multiple samples, and without intervention of an expert examiner.

The proposed system and method will fulfill these requirements because it will automatically centrifuge and stain with the Papanicolaou method (hematoxylin-eosin stain) in line with mandated medical best-practices for cancer detection. Many samples may be automatically examined at low cost by leveraging automatic image recognition software to fill roles conventionally occupied by expert examiners. In addition, the new system will standardize the performance of the examination and reproducibility of the results by using machine-consistent techniques to prepare the samples and identical examining methods between batches. This will allow medical professionals to compare current results with previous results with greater confidence in the reliability of the data, thereby enabling the monitoring of the evolution and progression of the disease over time. Finally, the proposed system may automatically identify malignant cells without the direct intervention of a pathologist. In practice, the data and determinations made by the present invention will be considered a screening test and not a histopathology procedure. The images of abnormal cells identified in the sediment will be automatically forwarded to a pathologist for accurate and final diagnosis, thereby maximizing the efficacy of a trained professional by elimination a large portion of the routine laboratory work required by conventional methods.

### BRIEF DESCRIPTION OF THE DRAWINGS

FIG. 1 is a schematic view of the system for the present invention.

FIG. 2 is a focused process view of the present invention.

FIG. 3 is an exemplary image of an analyte prepared via the overall process of the present invention, enhanced via microscope.

FIG. 4A is a flowchart illustrating the overall process of the present invention.

FIG. 4B is a continuation of FIG. 4A.

FIG. 5 is a flowchart illustrating a sub-process for acquiring and applying accurate labelling information to sample tubes.

FIG. 6 is a flowchart illustrating a sub-process for remotely moderating and executing a separation process.

FIG. 7 is a flowchart illustrating a sub-process for automatically extracting testable material post-separation.

FIG. 8 is a flowchart illustrating a sub-process for applying a chemical stain to slides, directly.

FIG. 9 is a flowchart illustrating a sub-process for applying a chemical stain to slides via immersion.

FIG. 10 is a flowchart illustrating a sub-process for automatically capturing visual data from a series of prepared slides.

FIG. 11 is a flowchart illustrating a sub-process for automatically performing an initial assessment of visual data.

FIG. 12 is a flowchart illustrating a sub-process for automatically applying a diagnosis of individual datum contained within visual data.

FIG. 13 is a flowchart illustrating a sub-process for automatically disseminating test results to preset recipients.

FIG. 14 is a flowchart illustrating a sub-process for assembling and collating multiple results of multiple consecutive iterations of the overall process.

### DETAILED DESCRIPTION OF THE INVENTION

All illustrations of the drawings are for the purpose of describing selected versions of the present invention and are not intended to limit the scope of the present invention. The present invention is to be described in detail and is provided in a manner that establishes a thorough understanding of the present invention. There may be aspects of the present invention that may be practiced or utilized without the implementation of some features as they are described. It should be understood that some details have not been described in detail in order to not unnecessarily obscure focus of the invention. References herein to “the preferred embodiment”, “one embodiment”, “some embodiments”, or “alternative embodiments” should be considered to be illustrating aspects of the present invention that may potentially vary in some instances, and should not be considered to be limiting to the scope of the present invention as a whole.

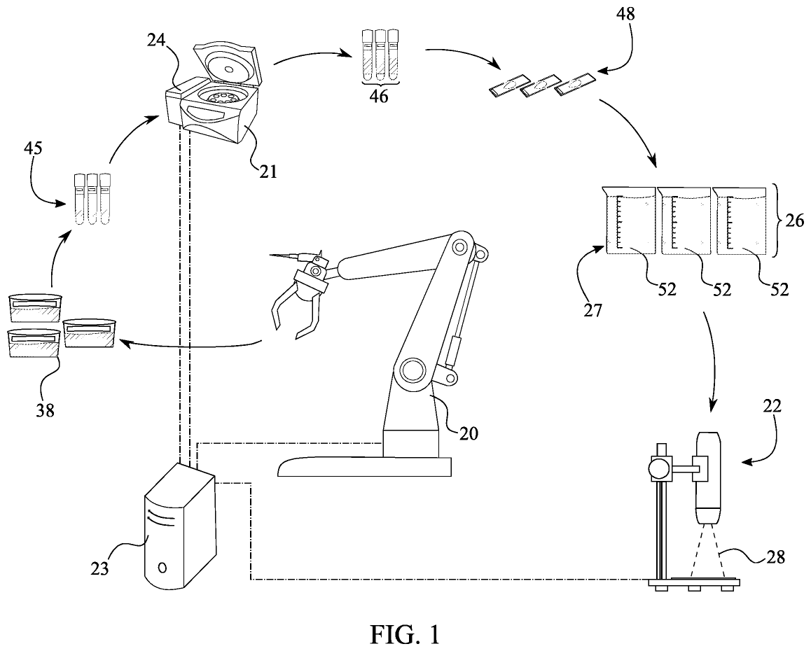

In reference to FIG. 1 through 14, the present invention is a system and method of processing and analyzing urine samples for identifying cancer cells. The automation of the preparation and initial examination processes and resultant reduction of manual tasks reduces variance between testing procedures as a natural consequence of human error, while simultaneously increasing the feasibility of frequent testing cycles as part of a more robust diagnostic regimen. To accomplish this, the system of the present invention utilizes at least one manipulator arm 20, at least one centrifuge 21, at least one electronic microscope 22, and at least one unitary controller 23 (Step A). The system of the present invention is further provided with at least one source sample 38. The source sample 38 is a volume of collected urine or another analyte to be processed by the present invention. The manipulator arm 20 is a conventional programmable robotic arm in the preferred embodiment, complete with a series of end-effectors and manipulator heads as may typically be found on such installations. Dedicated installations of other such robotic arms are known to be mounted to a mobile platform or other means of displacing the manipulator arm 20, particularly in implementations involving linearly arranged processing areas. In reference to FIG. 1, the manipulator arm 20 may be centrally positioned within reach of multiple such processing areas to enable a single instance of the manipulator arm 20 to facilitate all functions of the present invention. Further, at least one manipulator arm 20 may be multiple manipulator arms 20, wherein the instances of the manipulator arm 20 are arranged to engage all other elements of the present invention cooperatively.

The centrifuge 21 constitutes a conventional separation tool as may be readily available in laboratories and recognized by any suitable skilled individual. The centrifuge 21 is ideally configured for electronic control in communication with the unitary controller 23. More specifically, the operating parameters of the centrifuge 21 (rotations per minute, time in operation, start/stop commands, settle period) may be set and adjusted remotely via the unitary controller 23. Similarly, the electronic microscope 22 defines a conventional image enhancement tool that is communicably coupled to the unitary controller 23.

The electronic microscope 22 is preferably a bright light microscope with a ×10 low power field and a ×40 high power field configured to automatically capture and relay image data to the unitary controller 23 for analysis. Limitations to the type and power of the electronic microscope 22 should not be inferred from the preferred magnification power; any type of suitable magnifier or microscope may be supplemented without departing from the original spirit and scope of the present invention. Accordingly, the unitary controller 23 defines a centralized command and control system communicably coupled to the manipulator arm 20, the centrifuge 21, and the electronic microscope 22. The unitary controller 23 further defines a data processing hub suitable for image analysis and item recognition utilizing comparative analytical processes based on a cytopathological index contained therein. Additional functionalities related to out-processing of user-readable data are also supported within the unitary controller 23. The cytopathological index is a collection of interrelated reporting standards, classification thresholds, and exemplary image data that may be used to recognize, classify, and coherently describe abnormal cells captured by the microscope 22. ‘The Paris System for Reporting Urinary Cytology’ is one such element of the cytopathological index, providing a comprehensive set of terminology and diagnostic standards that may be used to effectively classify urothelial cells. Additional data may include, but is not limited to, bulk image data containing confirmed categories of atypical cells, actuarial tables relating to individual patient risk profiles, relevant medical history, or other data than may be used to inform and refine any diagnostic process performed by the present invention.

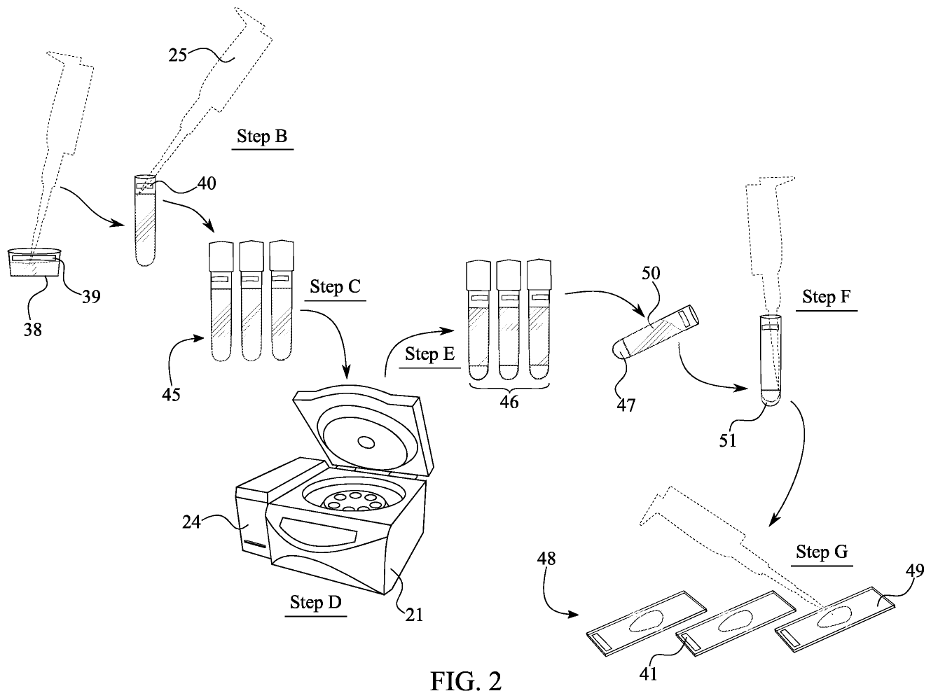

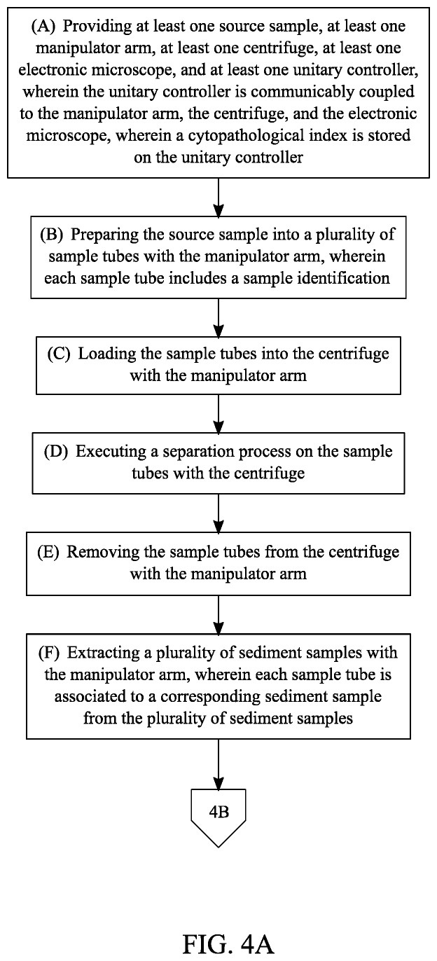

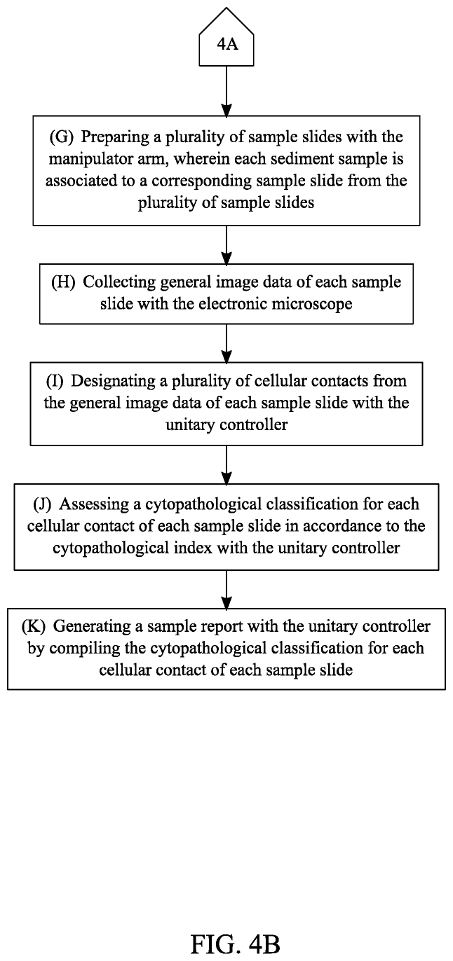

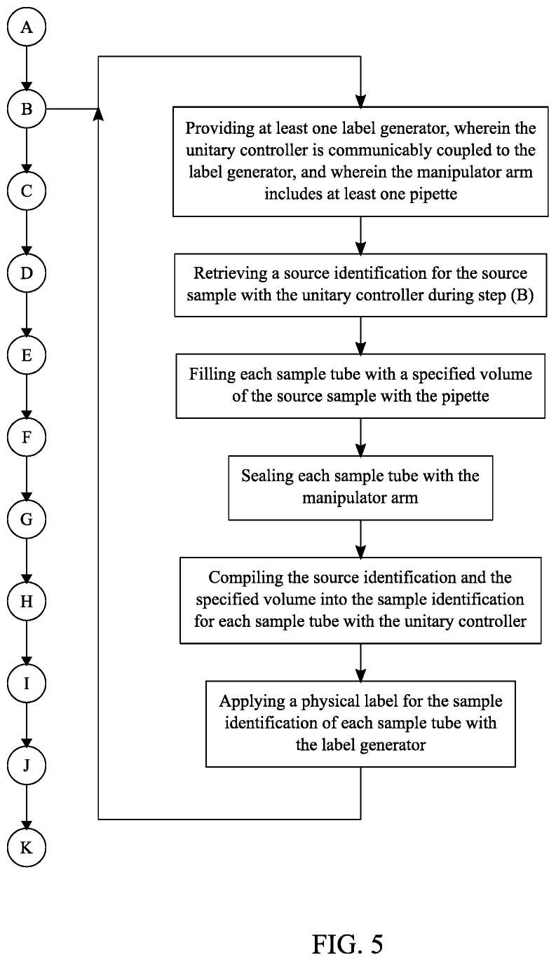

The overall process followed by the method of the present invention allows the aforementioned components of the system to automatically prepare, analyze, detect, and classify atypical cells by automating the chemical staining process and employing image recognition software. Referring to FIGS. 2, 4A, and 4B, the overall process begins by preparing the source sample 38 into a plurality of sample tubes 45 with the manipulator arm 20, wherein each sample tube 45 includes a sample identification 39 (Step B). The plurality of sample tubes 45 defines conventional centrifugation tubes compatible with the centrifuge 21, as previously outlined. The sample identification 39 constitutes a form of readable or scannable indicia fixed to each individual sample tube 45 to ensure that the plurality of sample tubes 45 associated with an arbitrary source sample 38 are not misplaced during the overall process. As outlined in FIG. 5, each sample identification 39 will define a printed physical label 40 automatically applied by the manipulator arm 20 utilizing adhesive. In another instance, the sample identification 39 may define a programmable identifier tag associated with each sample tube 45 and encoded within the unitary controller 23. Accordingly, the unitary controller 23 will catalogue all instances within the plurality of sample tubes 45 based on their association to an original source sample 38. Preparation of the source sample 38 constitutes the targeted selection and extraction of preconfigured volumes of analyte material into testable batches to enable a single source sample 38 to provide multiple rounds of test data. Consistency of testing standards between batches is enabled by digitizing the selection and extraction process utilizing the manipulator arm 20, thereby eliminating human error inherent to a conventional manual preparation process. Consequently, resultant data sets may be analyzed with a minimized margin for error and a reduced incidence of misdiagnosis stemming from repeatability errors.

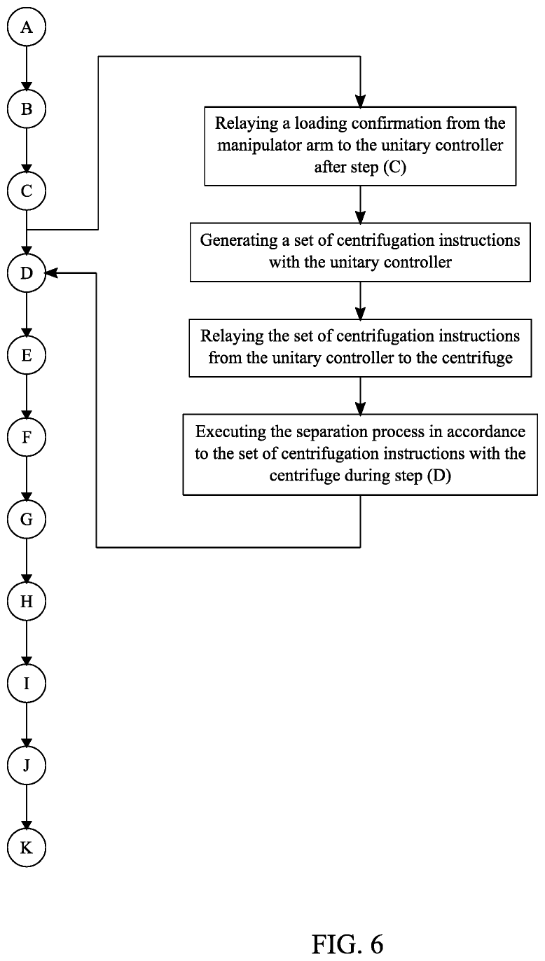

The overall process continues by loading the sample tubes 45 into the centrifuge 21 with the manipulator arm 20 (Step C), wherein the sample tubes 45 are individually seated within corresponding receptacles of the centrifuge 21 to ensure proper operation of the centrifuge 21. In reference to FIG. 6, once loading is complete the unitary controller 23 signals the centrifuge 21 to close and seal the operating hatch over the plurality of sample tubes 45 prior to beginning the centrifugation cycle. At least one implementation of the present invention may utilize a centrifuge 21 without an integral automatic closure mechanism. In this instance, the manipulator arm 20 may be configured to close the operating hatch.

Once secured, the overall process continues with executing a separation process on the sample tubes 45 with the centrifuge 21 (Step D). This separation process is defined as a centrifugation process, wherein the particles of the solution contained within the sample tubes 45 are separated according to size, shape, density, viscosity, and programmable rotor speed of the centrifuge 21. The operating parameters of the centrifuge 21 are stored within the unitary controller 23 as machine-readable instructions communicated to the centrifuge 21. Such operating parameters may include, but are not limited to, rotor speed, process duration, resting cycles, or any other metrics that may guide the execution of the separation process.

After the separation process is complete, the overall process continues by removing the sample tubes 45 from the centrifuge 21 with the manipulator arm 20 (Step E). This step is conducted as a reversal of the loading process, either individually removing sample tubes 45 or extracting an entire batch of a plurality of sample tubes 45 simultaneously before proceeding.

Subsequently, the overall process continues by extracting a plurality of sediment samples 46 with the manipulator arm 20, wherein each sample tube 45 is associated to a corresponding sediment sample 47 from the plurality of sediment samples 46 (Step F). The plurality of sediment samples 46 defines the collected testable particulate matter separated from the source sample 38 material during centrifugation. In the preferred implementation of the present invention, the plurality of sediment samples 46 will contain urothelial cells divisible into multiple diagnostic categories based on visually identifiable features. According to the internal documentation methods outlined thus far, the unitary controller 23 digitally associates the sediment sample 46 to the sample tube 45, then to the source sample 38 in a hierarchal format.

According to this hierarchal structure, the overall process continues by preparing a plurality of sample slides 48 with the manipulator arm 20, wherein each sediment sample 46 is associated to a corresponding sample slide 49 from the plurality of sample slides 48 (Step G). The plurality of sample slides 48 refers to a series of conventional transparent specimen carriers configured to mount within the field of view 28 of the electronic microscope 22. The association between sediment sample 46 and sample slide 48 may be denoted on each sample slide 48 with an additional printable tag or indicator to ensure that multiple batches of source samples 38 being processed through the system are not misidentified or cross-contaminated in later stages of operation.

The next stage of the overall process begins with collecting general image data 30 of each sample slide 48 with the electronic microscope 22 (Step H). This general image data 30 defines a relatively low-magnification view of the target sample slide 48 suitable for cursory analysis and processing to determine areas of interest for more intensive imaging and analysis in later steps.

Accordingly, the overall process continues by designating a plurality of cellular contacts 31 from the general image data 30 of each sample slide 48 with the unitary controller 23 (Step I). The plurality of cellular contacts 31 defines a machine-generated list of possible zones within the general image data 30 that are identified as containing urothelial cells and therefore may require more investigation to determine malignancy. This analysis is performed by the unitary controller 23, wherein the unitary controller 23 serves as a graphics processing unit.

The intensive imaging and investigation processes are subsequently associated with assessing a cytopathological classification for each cellular contact of each sample slide 48 in accordance to the cytopathological index with the unitary controller 23 (Step J). The cytopathological index defines a uniform machine-readable series of thresholds for identifying and reporting urothelial cells based on visually observable characteristics, i.e. size, shape, opacity, geometric complexity, or other standards as may be known to a reasonably skilled individual. The cytopathological classification for each cellular contact is thus a uniform reporting code for a profile defined by the cytopathological index, ideally categorizing each cellular contact as ‘healthy’, ‘benign’, ‘malignant’, or possibly ‘unknown’ if no suitable classification may be attached. These categories are exemplary of a preferred implementation; however, imitations to the type and descriptors of the classifications should not be implied.

The overall process concludes by generating a sample report with the unitary controller 23 by compiling the cytopathological classification for each cellular contact of each sample slide 48 (Step K). The sample report ideally contains a tabulation of all identifiable cellular contacts 31, including adjacent copies of sample reports generated for previous batches of source sample 38. The rendering of this data may include graphical representations of the volume of each cytopathological classification detected within multiple sequential batches to aid in a diagnosis of disease progression over time. The sample report may additionally include a preliminary machine-generated diagnosis based on the assessed presence and levels of various classifications within a given source sample 38.

Referring to FIG. 5, one subprocess for the method of the present invention is used to properly label and prepare the plurality of sample tubes 45 to prevent misattribution of testing results across multiple concurrent batches or patients. Thus, the subprocess is provided with at least one label generator 24, wherein the unitary controller 23 is communicably coupled to the label generator 24, and wherein the manipulator arm 20 includes at least one pipette 25. The label generator 24 may define any form of printer, laser engraver, or configurable ink stamp that may visually mark lab materials. The unitary controller 23 provides operating instructions to the label generator 24, as with the centrifuge 21 and manipulator arm 20, to ensure effective cooperation between the disparate hardware comprising the system of the present invention. In addition, the manipulator arm 20 includes at least one pipette 25 in this instance. The pipette 25 constitutes a means of extracting a programmable volume of liquid, including any solute or suspended particulate material in said volume. To enable rapid processing of large quantities of testable material and maintain tool sterility, at least one pipette 25 may be positioned in a battery arrangement and/or equipped with self-sterilization functions as may be readily apparent to an individual skilled in the art. This subprocess begins by retrieving a source identification from the source sample 38 with the unitary controller 23 during Step B. The source identification defines both a printed indicator and a digital counterpart to the printed indicator that are unique to the source sample 38. In one instance, the manipulator arm 20 may facilitate the acquisition of the source identification from the source sample 38 with an integrated visual scanner or equivalent article. This subprocess continues by filling each sample tube 45 with a specified volume of the source sample 38 with the pipette 25. The specified volume refers to a testable amount of analyte as defined within the unitary controller 23. As previously outlined, the pipette 25 is directed to extract a consistent amount of analyte per operation to ensure that testing protocols and standards are maintained across multiple iterations of testing. This subprocess continues by sealing each sample tube 45 with the manipulator arm 20. The manipulator arm 20 is typically considered to utilize conventional caps or stoppers for this function, but any form of impermeable seal may be applied here. The unitary controller 23 subsequently compiles the source identification and the specified volume into the sample identification 39 for each sample tube 45, wherein the data corresponding to each of these distinct entries is attached to a retrievable record within the unitary controller 23. The collection of all testing data in this manner may enable the review and analysis of procedures to ensure adherence to test protocols in conjunction with a larger quality assurance program and established practices within the field. In accordance with this objective, the label generator 24 applies a physical label 40 for the sample identification 39 of each sample tube 45. The physical label 40 enables a sample to be manually tracked through the method of the present invention as a secondary measure in conjunction with the integrated tracking elements of the unitary controller 23. In practice, this feature enables the periodical spot-checking of the present invention by comparing the information written to the physical label 40 to the corresponding entries in the unitary controller 23.

Referring to FIG. 6, another subprocess enables the digitization of the operation of the centrifuge 21. This subprocess begins by relaying a loading confirmation from the manipulator arm 20 to the unitary controller 23 after Step C. The loading confirmation constitutes a digital signal produced at the centrifuge 21 upon closure of an access panel or lid, verifying that a complete load of the plurality of sample tubes 45 is properly mounted within the centrifuge 21. Subsequently, the appropriate separation operation must be executed according to presets stored within the unitary controller 23, which is accomplished by generating a set of centrifugation instructions with the unitary controller 23 then relaying the set of centrifugation instructions from the unitary controller 23 to the centrifuge 21. The set of centrifugation instructions may constitute a single data package of executable instructions readable in series by the centrifuge 21 or may be delivered in sequence by the unitary controller 23 dependent on the instruction buffer of the centrifuge 21. The overall process continues by executing the separation process in accordance to the set of centrifugation instructions with the centrifuge 21 during Step D. The unitary controller 23 will ideally moderate the separation process to ensure consistent and effective operation of the centrifuge 21 per testing standards. As outlined previously, the unitary controller 23 may remotely adjust rotations per minute, time in operation, start/stop commands, settle periods, or any other aspect of centrifugation as may be realized by a reasonably skilled individual.

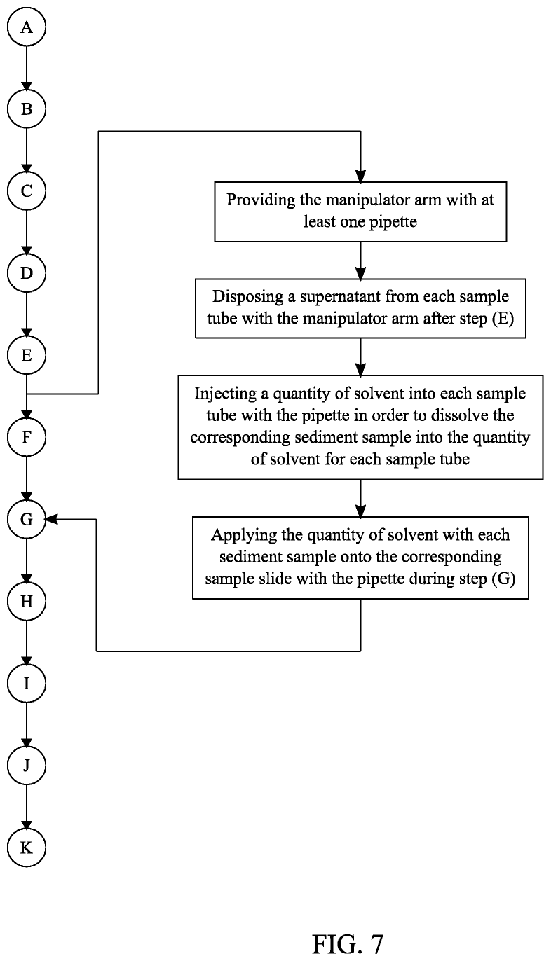

Referring to FIG. 7, another subprocess allows waste material to be removed post-centrifugation prior to rendering the sediment samples 46 for inspection. This subprocess provides the manipulator arm 20 with at least one pipette 25, wherein the pipette 25 disposes of a supernatant 50 from each sample tube 45 with the manipulator arm 20 after Step E. The supernatant 50 conventionally describes any post-separation liquid residue in the plurality of sample tubes 45. This supernatant 50 is distinct from the sediment samples 46, wherein the sediment samples 46 are retained for processing and inspection as outlined previously. Next, this subprocess continues by injecting a quantity of solvent 51 into each sample tube 45 with the pipette 25 in order to dissolve the corresponding sediment sample 47 into the quantity of solvent 51 for each sample tube 45. More specifically, a known quantity of solvent 51 is applied to each of the plurality of sample tubes 45 to ensure that the entirety of each sediment sample 46 is removed from the corresponding sample tube 45. Rendering the sediment sample 46 and the solvent 51 as a composite solution enables the lossless transfer of the sediment sample 46 to a subsequent media. Accordingly, this subprocess concludes by applying the quantity of solvent 51 with each sediment sample 46 onto the corresponding sample slide 49 with the pipette 25 during Step G.

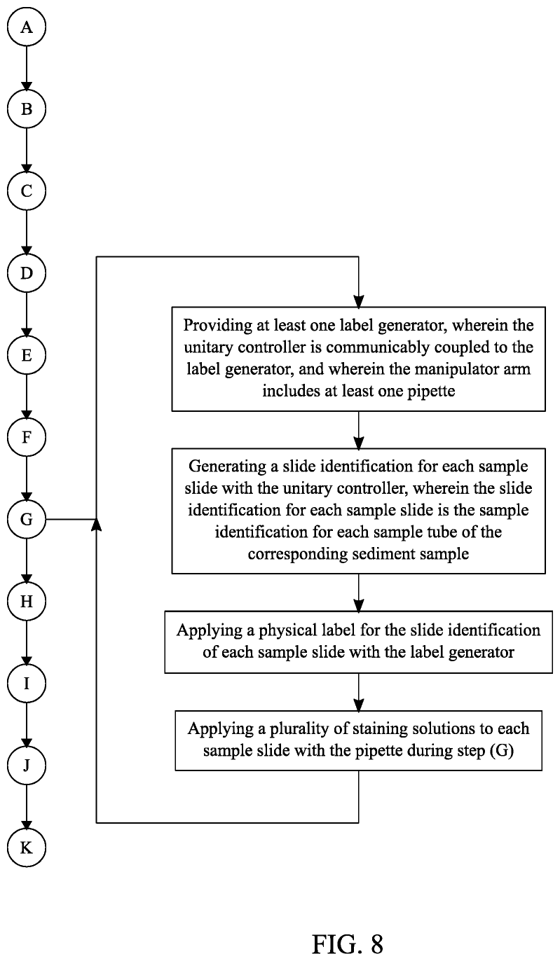

Referring to FIG. 8, another subprocess allows the sediment samples 46 to be chemically stained with a pipette 25. This subprocess is provided with at least one label generator 24, wherein the unitary controller 23 is communicably coupled to the label generator 24, and wherein the manipulator arm 20 includes at least one pipette 25. The subprocess continues by applying a physical label 40 for the slide identification 41 of each sample slide 48 with the label generator 24, similar to the subprocess by which a similar label may be applied to the individual sample tubes 45, previously. By extending the labelling process across all media, the integrity of any testable batches may be assured. In the event of misplacement, disordering, or loss of any testable media, the unitary controller 23 may register the loss and alert operators as appropriate. In this subprocess, chemical stains are applied directly by the manipulator arm 20 via the pipette 25. Specifically, this subprocess continues by applying a plurality of staining solutions 52 to each sample slide 48 with the pipette 25 during Step G. The plurality of staining solutions 52 is specifically contemplated to comprise acetic acid, water, OG-6 dye, EA-50 dye, methanol, and xylene in variable concentrations and order. The conventional Papanicolaou (“Pap”) stain process is considered useful for exemplary purposes, however, the specific composition and application of the plurality of staining solutions 52 is suggested to be variable across multiple embodiments without departing from the original spirit and scope of the present invention.

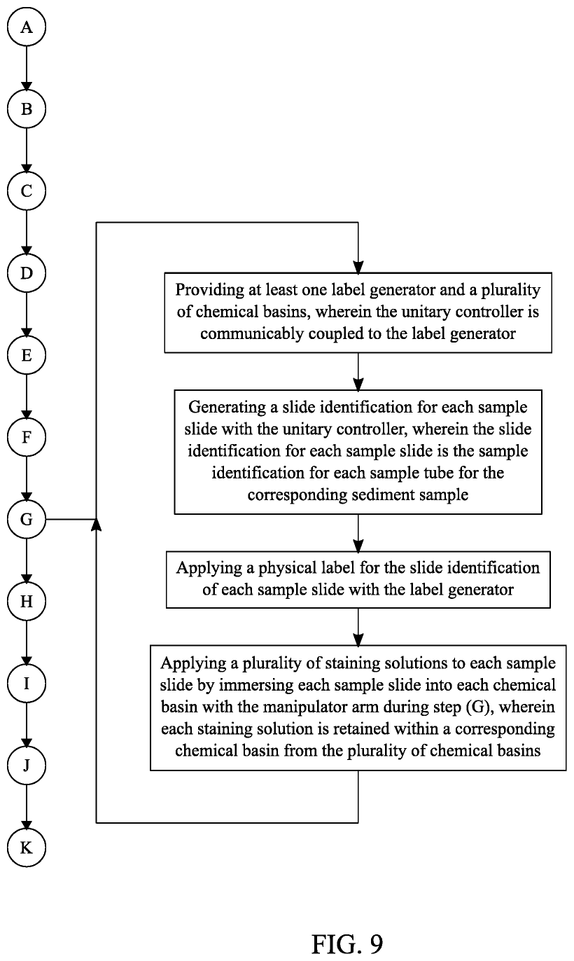

As an alternative to the previous subprocess, another subprocess allows the sediment samples 46 to be chemically stained with multiple bathing basins 26. This subprocess is provided with at least one label generator 24 and a plurality of chemical basins 26, wherein the unitary controller 23 is communicably coupled to the label generator 24. Similar to the previous subprocess, this subprocess begins by generating a slide identification 41 for each sample slide 48 with the unitary controller 23, wherein the slide identification 41 for each sample slide 48 is the sample identification 39 for each sample tube 45 for the corresponding sediment sample 47. Again, similar to the previous subprocess, this subprocess continues by applying a physical label 40 for the slide identification 41 of each sample slide 48 with the label generator 24 as outlined in FIG. 9. Consistent labelling throughout the overall process is considered essential to prevent misattribution of test results between patients. A simple error at this stage may have far-reaching effects on ultimate patient outcomes if the data used to inform treatment decisions is presented erroneously. The manipulator arm 20 then applies a plurality of staining solutions 52 to each sample slide 48 by immersing each sample slide 48 into each chemical basin with the manipulator arm 20 during Step G, wherein each staining solution is retained within a corresponding chemical basin 27 from the plurality of chemical basins 26. The plurality of chemical basins 26 ideally defines a series of segmented liquid containers of appropriate dimensions to accept the plurality of sample slides 48, internally. This arrangement also includes any internal supporting structures or retainers that may capture the individual sample slide 48 during the staining process. In an ideal arrangement, the plurality of chemical basins 26 is aligned according to the preferred application order of the staining process. In this arrangement, the corresponding chemical basin 27 is always immediately adjacent to the sample slide 48 slated for immersion in said corresponding chemical basin 27.

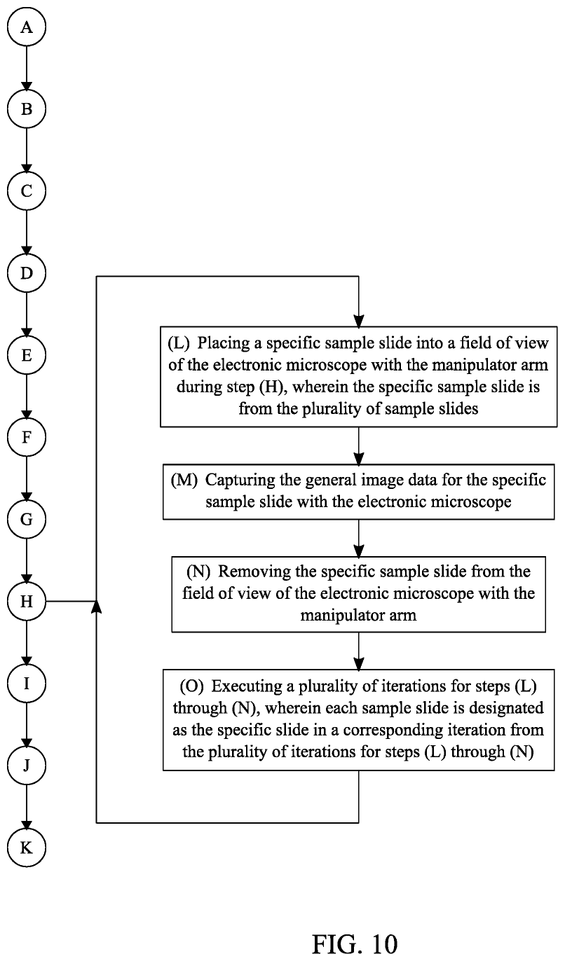

Another subprocess allows for automatic visual analysis the is supported by dedicated hardware suitable for automation (e.g. the electronic microscope 22). In reference to FIG. 10, this subprocess begins by placing a specific sample slide 48 into a field of view 28 of the electronic microscope 22 with the manipulator arm 20 during Step H, wherein the specific sample slide 48 is from the plurality of sample slides 48 (Step L). The field of view 28 is physically defined as the targeted focal point of the electronic microscope 22 and is digitally defined by the unitary controller 23 as a transformation of the position of the manipulator arm 20. More specifically, the field of view 28 may define an origin from which movement orders are defined, enabling repeated returns to this origin point as both an essential function of the overall system and an error mitigation method. After the specific sample slide 48 is positioned within the field of view 28, this subprocess continues by capturing the general image data 30 for the specific sample slide 48 with the electronic microscope 22 (Step M). The general image data 30 constitutes a broad overview of the specific sample slide 48 and the corresponding sediment sample 47 contained therein for the purposes of establishing an initial diagnosis. The initial diagnosis generated by the unitary controller 23 catalogues items of interest within the general image data 30 for later analysis, ideally performed based on a triage of the items of interest performed at this stage. This subprocess continues by removing the specific sample slide 48 from the field of view 28 of the electronic microscope 22 with the manipulator arm 20 (Step N) to clear the field of view 28 for a subsequent sample slide 48. Accordingly, this subprocess is repeated by executing a plurality of iterations for Step L through Step N, wherein each sample slide 48 is designated as the specific slide in a corresponding iteration from the plurality of iterations for Step L through Step N (Step O). This reiteration is continued for the entire plurality of sample slides 48, until all sediment samples 46 are catalogued with general image data 30 corresponding to each specific sample slide 48.

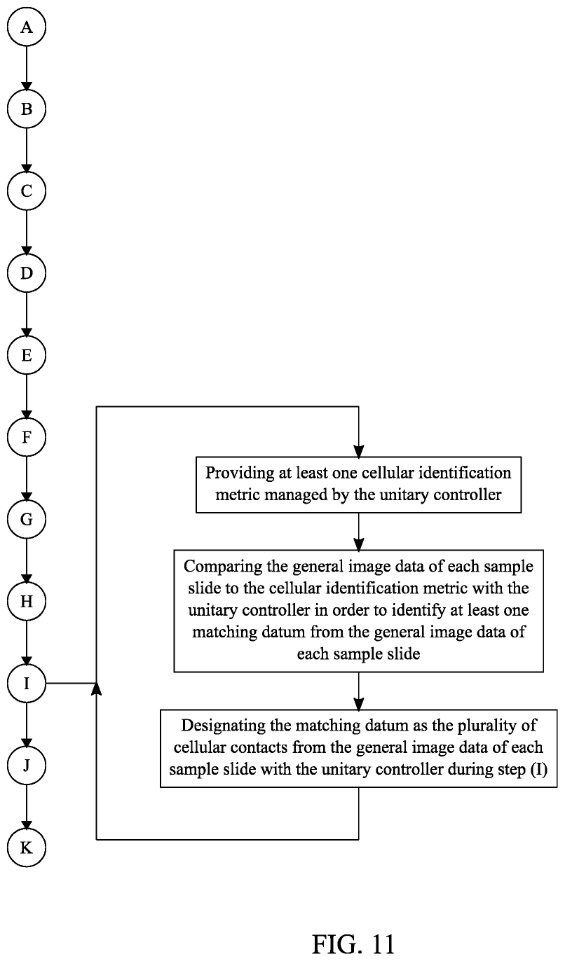

Another subprocess allows for the automatic visual identification of medically significant cellular conditions, as outlined in FIG. 11. This subprocess is provided with at least one cellular identification metric that is managed by the unitary controller 23. The cellular identification metric defines an established standard for differentiating between various archetypes of a cell based on visual characteristics. In various conceivable applications, any singular cellular identification metric may be applied in conjunction with multiple distinct cellular identification metrics to generate a robust diagnosis based on established confidence thresholds for said visual characteristics. As outlined in FIG. 11, this subprocess begins by comparing the general image data 30 of each sample slide 48 to the cellular identification metric with the unitary controller 23 in order to identify at least one matching datum from the general image data 30 of each sample slide 48. The matching datum defines any characteristic determined to be within the thresholds for positive inclusion to any class or category of cellular contact. Accordingly, this subprocess continues by designating the matching datum as the plurality of cellular contacts 31 from the general image data 30 of each sample slide 48 with the unitary controller 23 during Step I. In practice, the matching datum is compiled with other instances of matching datum and utilized to compile a list of inclusive categories, whereby said categories may be weighted during analysis to determine final placement of the corresponding cellular contact into a simplified classification. This simplified classification, or preliminary diagnosis, is attached to each individual cellular contact within the unitary controller 23. Consequently, each cellular contact may be reviewed within an accessible database as opposed to manually reacquiring said cellular contact within an archived sample slide 48 or article of general image data 30.

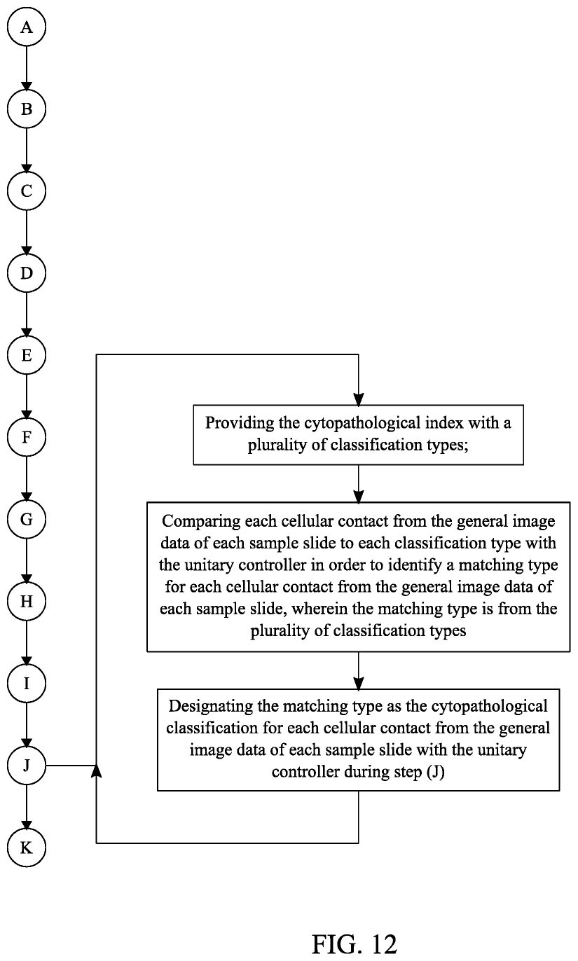

After general image data 30 is captured for each specific sample slide 48, another subprocess allows for in-depth analysis to extract actionable data from the broader data sets outlined previously. The actionable data is defined in relation to a reporting standard for cellular contacts 31, specifically urothelial cells and visual characteristics thereof. Conventional laboratory testing requires a technician to visually identify these urothelial cells post-stain to determine the type and quantity of cellular contacts 31 present within a sample. Referring to FIG. 12, provisions are made to enable the unitary controller 23 to automatically apply these standards in order to provide the cytopathological index with a plurality of classification types. These classification types may be attached to more simplistic monikers for ease of use, such as ‘healthy’, ‘benign’, ‘malignant’, or other labels as previously outlined. However, the plurality of classification types is understood to encompass all identifiable characteristics of cells as may be visually ascertained, including a detailed description of these characteristics behind any simplified labels to be stored within the unitary controller 23. This subprocess begins by comparing each cellular contact from the general image data 30 of each sample slide 48 to each classification type with the unitary controller 23 in order to identify a matching type for each cellular contact from the general image data 30 of each sample slide 48, wherein the matching type is from the plurality of classification types. Automatic identification and classification of each cellular contact enables the present invention to perform diagnostic processes from initial sample preparation through data interpretation, minimizing the manpower requirements for routine testing. Further, the digitization of the plurality of classification types enables the collection of training data that may be used to refine the automatic identification processes in an iterative learning environment. This subprocess finishes by designating the matching type as the cytopathological classification for each cellular contact from the general image data 30 of each sample slide 48 with the unitary controller 23 during step J. This refinement will enable a diagnostician or attending physician to rapidly assess the cellular contacts 31 present within the general image data 30, consequently accelerating the testing procedure and removing barriers to additional testing that may otherwise render such efforts prohibitive.

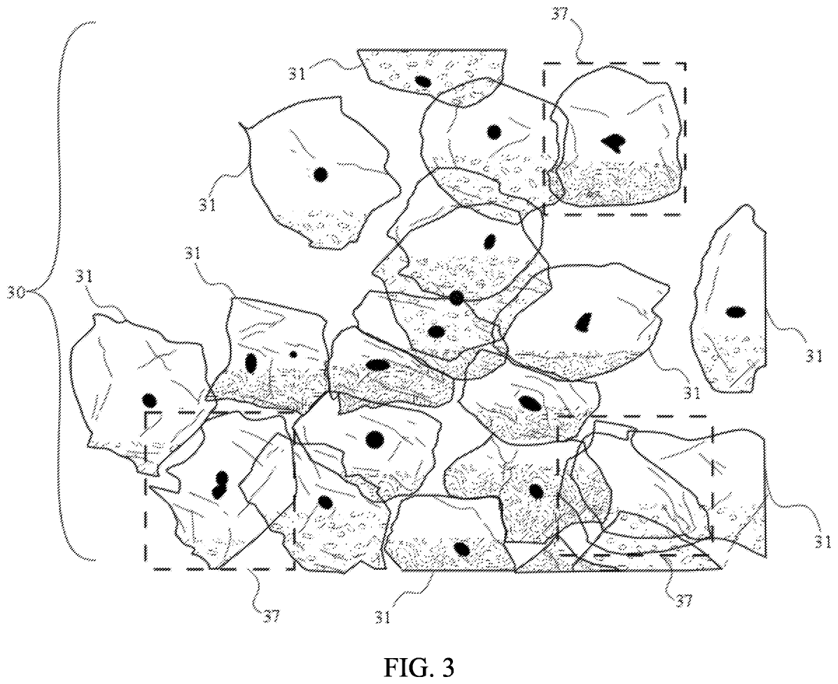

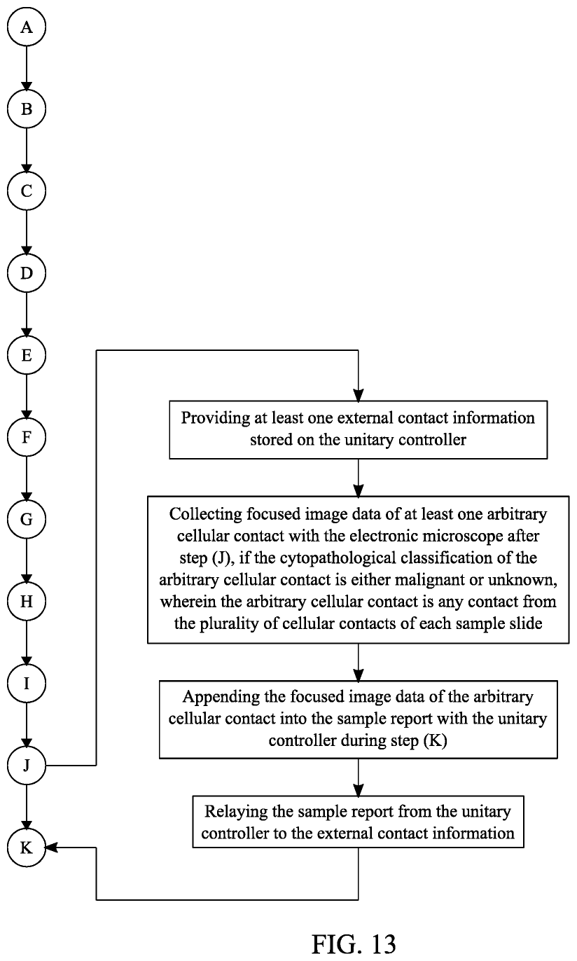

Another subprocess allows the present invention to disseminate data to medical professionals in an effort to collaboratively diagnose a patient using said data. In this embodiment, the present invention may integrate opinions and recommendations from external sources to modify and improve the automatic image recognition system at the core of the present invention. As outlined in FIG. 13, this subprocess is provided with at least one external contact information stored on the unitary controller 23. The external contact information may be an email address, phone number, or internal identifier for any telemedicine software application compatible with the unitary controller 23. The external contact information is preferably prerecorded within the unitary controller 23 as ancillary data attached to the source sample 38, identifying a supervising physician or person otherwise responsible for applying the test results in a broader context. As shown in exemplary form in FIG. 3, this subprocess continues by collecting focused image data 37 of at least one arbitrary cellular contact with the electronic microscope 22 after Step J, if the cytopathological classification of the arbitrary cellular contact is either malignant or unknown, wherein the arbitrary cellular contact is any contact from the plurality of cellular contacts 31 of each sample slide 48. The focused image data 37 defines localized image data centered around cellular contacts 31 determined to fall within categorizations other than ‘healthy’ during previous analyses of the general image data 30. This subprocess generally presumes that cellular contacts 31 recognized as non-problematic with a high degree of confidence need not be targeted for additional investigation, though the focused image data 37 may be captured by direction or policy as configured by a user. Separating each cellular contact by location within the general image data 30 enables the electronic microscope 22 to target the arbitrary cellular contact with higher magnification, enabling the unitary controller 23 to capture and analyze the arbitrary cellular contact for characteristics that may have been obscured or otherwise unclear in the general image data 30. Once the focused image data 37 relating to the arbitrary cellular contact is captured and analyzed, this subprocess concludes by appending the focused image data 37 of the arbitrary cellular contact into the sample report with the unitary controller during Step K and then relaying the sample report from the unitary controller 23 to the external contact information. The pre-filtering of image data serves to reduce time spent on non-critical analyses, freeing additional time for in-demand medical professionals to perform in-depth analyses as required. Further, the automatic and direct presentation of said image data reduces organizational inefficiencies stemming from lost or non-standard communiques between medical and administrative staff.

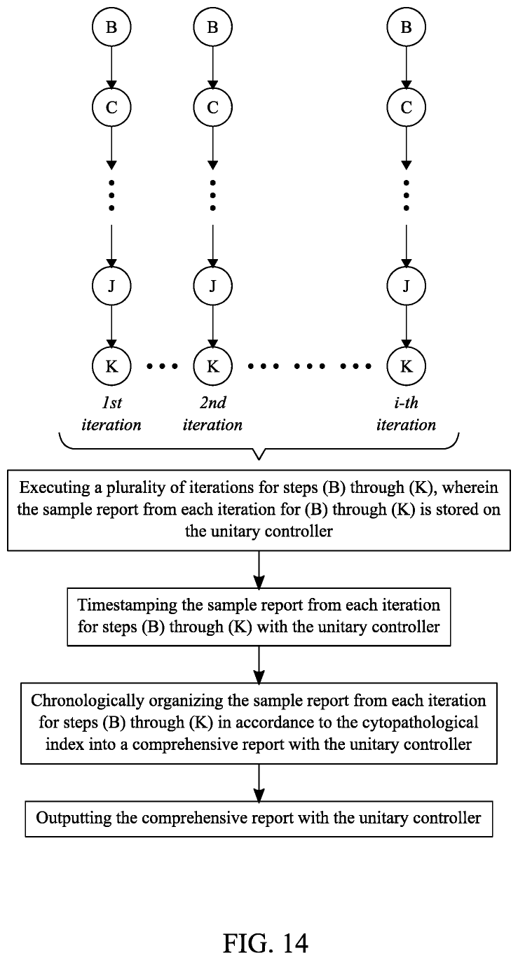

The present invention is proposed to be beneficial as both a standalone process and a tool for generating consistent time-scaled diagnostics and assessments of extended courses of treatment. As shown in FIG. 14, a plurality of iterations for Step B through Step K are executed so that the sample report from each iteration for Step B through Step K is stored on the unitary controller 23. The sample report is retained by the unitary controller 23 for continuous comparison to future iterations of the overall process, whereby insights into disease progression and treatment efficacy may be gained from a record. Supporting this file arrangement, the unitary controller 23 timestamps the sample report from each iteration for Step B through Step K and then chronologically organizes the sample report from each iteration for Step B through Step K in accordance to the cytopathological index into a comprehensive report. Appending an indelible time reference to the sample report enables all data contained within the sample report to be automatically modelled over time by conventional data handling software. In the preferred embodiment, the comprehensive report includes an evolving data set stored within the unitary controller 23. This comprehensive report supports the inclusion of all historical medical data (physicians notes, observational reports, admission records, toxicology screens, etc.) as contemporaneous appendages to the data generated in relation to the plurality of cellular contacts 31. Utilizing this data, a physician can expect to map trends in patient condition to forecast future developments. Though the comprehensive report is preferably contained by the unitary controller 23, it is suggested that multiple instances of the comprehensive report corresponding to a single patient may be maintained remotely via persistent updates from the unitary controller 23. Finally, the unitary controller 23 outputs the comprehensive report, wherein an output of the comprehensive report may be digital and/or physical copies of the conclusions and supporting data.

Although the invention has been explained in relation to its preferred embodiment, it is to be understood that many other possible modifications and variations can be made without departing from the spirit and scope of the invention as hereinafter claimed.

What is claimed is:

1. A method of automatically preparing and analyzing urine samples for identifying cancer cells, the method comprises the steps of:
(A) providing at least one source sample, at least one manipulator arm, at least one centrifuge, at least one electronic microscope, and at least one unitary controller, wherein the unitary controller is communicably coupled to the manipulator arm, the centrifuge, and the electronic microscope, wherein a cytopathological index is stored on the unitary controller;
(B) preparing the source sample into a plurality of sample tubes with the manipulator arm, wherein each sample tube includes a sample identification;
(C) loading the sample tubes into the centrifuge with the manipulator arm;
(D) executing a separation process on the sample tubes with the centrifuge;
(E) removing the sample tubes from the centrifuge with the manipulator arm;
(F) extracting a plurality of sediment samples with the manipulator arm, wherein each sample tube is associated to a corresponding sediment sample from the plurality of sediment samples;
(G) preparing a plurality of sample slides with the manipulator arm, wherein each sediment sample is associated to a corresponding sample slide from the plurality of sample slides;
(H) collecting general image data of each sample slide with the electronic microscope;
(I) designating a plurality of cellular contacts from the general image data of each sample slide with the unitary controller;
(J) assessing a cytopathological classification for each cellular contact of each sample slide in accordance to the cytopathological index with the unitary controller; and
(K) generating a sample report with the unitary controller by compiling the cytopathological classification for each cellular contact of each sample slide.

2. The method of automatically preparing and analyzing urine samples for identifying cancer cells, the method as claim 1 comprises the steps of:
providing at least one label generator, wherein the unitary controller is communicably coupled to the label generator, and wherein the manipulator arm includes at least one pipette;
retrieving a source identification for the source sample with the unitary controller during step (B);
filling each sample tube with a specified volume of the source sample with the pipette;
sealing each sample tube with the manipulator arm;
compiling the source identification and the specified volume into the sample identification for each sample tube with the unitary controller; and
applying a physical label for the sample identification of each sample tube with the label generator.

3. The method of automatically preparing and analyzing urine samples for identifying cancer cells, the method as claim 1 comprises the steps of:
relaying a loading confirmation from the manipulator arm to the unitary controller after step (C);
generating a set of centrifugation instructions with the unitary controller;
relaying the set of centrifugation instructions from the unitary controller to the centrifuge; and
executing the separation process in accordance to the set of centrifugation instructions with the centrifuge during step (D).

4. The method of automatically preparing and analyzing urine samples for identifying cancer cells, the method as claim 1 comprises the steps of:
providing the manipulator arm with at least one pipette;
disposing a supernatant from each sample tube with the manipulator arm after step (E);
injecting a quantity of solvent into each sample tube with the pipette in order to dissolve the corresponding sediment sample into the quantity of solvent for each sample tube; and
applying the quantity of solvent with each sediment sample onto the corresponding sample slide with the pipette during step (G).

5. The method of automatically preparing and analyzing urine samples for identifying cancer cells, the method as claim 1 comprises the steps of:
providing at least one label generator, wherein the unitary controller is communicably coupled to the label generator, and wherein the manipulator arm includes at least one pipette;
generating a slide identification for each sample slide with the unitary controller, wherein the slide identification for each sample slide is the sample identification for each sample tube of the corresponding sediment sample;
applying a physical label for the slide identification of each sample slide with the label generator; and
applying a plurality of staining solutions to each sample slide with the pipette during step (G).

6. The method of automatically preparing and analyzing urine samples for identifying cancer cells, the method as claim 1 comprises the steps of:
providing at least one label generator and a plurality of chemical basins, wherein the unitary controller is communicably coupled to the label generator;
generating a slide identification for each sample slide with the unitary controller, wherein the slide identification for each sample slide is the sample identification for each sample tube for the corresponding sediment sample;
applying a physical label for the slide identification of each sample slide with the label generator; and
applying a plurality of staining solutions to each sample slide by immersing each sample slide into each chemical basin with the manipulator arm during step (G), wherein each staining solution is retained within a corresponding chemical basin from the plurality of chemical basins.

7. The method of automatically preparing and analyzing urine samples for identifying cancer cells, the method as claim 1 comprises the steps of:
(L) placing a specific sample slide into a field of view of the electronic microscope with the manipulator arm during step (H), wherein the specific sample slide is from the plurality of sample slides;
(M) capturing the general image data for the specific sample slide with the electronic microscope;
(N) removing the specific sample slide from the field of view of the electronic microscope with the manipulator arm; and
(O) executing a plurality of iterations for steps (L) through (N), wherein each sample slide is designated as the specific slide in a corresponding iteration from the plurality of iterations for steps (L) through (N).

8. The method of automatically preparing and analyzing urine samples for identifying cancer cells, the method as claim 1 comprises the steps of:
providing at least one cellular identification metric managed by the unitary controller;
comparing the general image data of each sample slide to the cellular identification metric with the unitary controller in order to identify at least one matching datum from the general image data of each sample slide; and
designating the matching datum as the plurality of cellular contacts from the general image data of each sample slide with the unitary controller during step (I).

9. The method of automatically preparing and analyzing urine samples for identifying cancer cells, the method as claim 1 comprises the steps of:
providing the cytopathological index with a plurality of classification types;
comparing each cellular contact from the general image data of each sample slide to each classification type with the unitary controller in order to identify a matching type for each cellular contact from the general image data of each sample slide, wherein the matching type is from the plurality of classification types; and
designating the matching type as the cytopathological classification for each cellular contact from the general image data of each sample slide with the unitary controller during step (J).

10. The method of automatically preparing and analyzing urine samples for identifying cancer cells, the method as claim 1 comprises the steps of:
providing at least one external contact information stored on the unitary controller;
collecting focused image data of at least one arbitrary cellular contact with the electronic microscope after step (J), if the cytopathological classification of the arbitrary cellular contact is either malignant or unknown, wherein the arbitrary cellular contact is any contact from the plurality of cellular contacts of each sample slide;
appending the focused image data of the arbitrary cellular contact into the sample report with the unitary controller during step (K); and
relaying the sample report from the unitary controller to the external contact information.

11. The method of automatically preparing and analyzing urine samples for identifying cancer cells, the method as claim 1 comprises the steps of:
executing a plurality of iterations for steps (B) through (K), wherein the sample report from each iteration for (B) through (K) is stored on the unitary controller;
timestamping the sample report from each iteration for steps (B) through (K) with the unitary controller;
chronologically organizing the sample report from each iteration for steps (B) through (K) in accordance to the cytopathological index into a comprehensive report with the unitary controller; and
outputting the comprehensive report with the unitary controller.
